(19) United States
(12) Patent Application Publication
(10) Pub. No.: US 2005/0110950 A1
(43) Pub. Date: May 26, 2005

(54) Saccadic motion sensing

(76) Inventors: William P. Thorpe, Winchester, MA (US); Charles P. Plant, Belmont, MA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/797,882

(22) Filed: Mar. 10, 2004

Related U.S. Application Data
(60) Provisional application No. 60/454,256, filed on Mar. 13, 2003.

(51) Int. Cl. A61B 3/14

(52) U.S. Cl. 351/209

(57) ABSTRACT

A saccadic-motion detector includes an optical apparatus configured to focus light received from a subject's eye onto a focal plane, and an optical navigation chip comprising an optical sensing surface disposed substantially in the focal plane of the optical apparatus, the optical navigation chip configured to convert analog light reflected from the eye to digital indicia of movement of the eye.

### CROSS-REFERENCE TO RELATED ACTIONS

This application claims the benefit of U.S. Provisional Application No. 60/454,256 filed Mar. 13, 2003.

### FIELD OF THE INVENTION

The invention relates to motion detection and more particularly to detection of saccadic eye motion.

### BACKGROUND OF THE INVENTION

Day surgery under general anesthesia is now common and continues to grow in popularity owing to patient convenience and medico-economic pressures. Perioperative care improvements have allowed surgeons to perform more invasive day-surgical procedures. Additionally, the availability of drugs, such as propofol and remifentanil, has improved anesthesia care for more extensive surgeries. After surgery, especially day surgery, patients usually desire discharge as soon as possible.

Saccadic eye movements can be used to monitor recovery from general anesthesia. Indeed, evaluation of saccadic eye movements is more sensitive than choice-reaction tests in detecting the residual effects of anesthesia. Also, evaluation of saccadic eye movements is more reliable than subjective state-of-alertness tests, such as the visual analogue score for sedation, owing to the tendency for subjects to underestimate their impairment.

Eye movements may be affected by alcohol or other drugs. The effects of alcohol on saccadic eye movements were reported over three decades ago. Indeed, in field sobriety tests, law enforcement officers are trained to recognize end-point nystagmus that can be elicited by having the subject gaze laterally to the extreme. At least one study has described the change in saccadic eye movements as a measure of drug induced CNS depression caused by Valium (diazepam). In the mid-1980s, the effects barbiturates, benzodiazepines, opiates, carbamazepine, amphetamine and ethanol on saccadic eye movements were observed using a computer system coupled to a television monitor that provided visual stimulation for the subject, and an electrooculogram that measured eye movements. Not surprisingly, barbiturates, benzodiazepines, opiates, carbamazepine, and ethanol reduced peak saccadic velocity while amphetamine increased it. Saccadic eye movements have been studied in subjects who were given nitrous oxide or isoflurane. Isoflurane caused significant diminution of mean saccadic peak velocity. In contrast, there was little effect caused by nitrous oxide or isoflurane on subjective assessment, assessed by subject's reporting of odor, tiredness, drowsiness, sleepiness, or nausea. It has also been reported that both cyclopropane and halothane depressed peak velocity of saccadic eye movements in a dose-dependent fashion. Peak saccadic velocity returned to baseline within 5 minutes after discontinuation. As found with isoflurane, no significant difference was found between halothane, cyclopropane, and air in subjective assessment of impairment. In a separate placebo-controlled trial, a diminution was found in peak saccadic velocity after propofol infusion. It has been suggested that a combination of peak saccadic velocity, percentage error and choice reaction time would be a potentially useful battery of tests to assess recovery from anesthesia.

More recently, the effect of isoflurane has been studied regarding (1) saccadic latency and (2) a countermanding task. In a saccadic latency test, a moving target comprising a light-emitting diode was displayed on a screen. The latency of eye movements after target movements was measured, and was found to increase with anesthetic dose. In the countermanding task, which requires a higher level of conscious performance, the subject was asked to voluntarily suppress gaze movement to the target. Again, anesthetic increased the latency of response. Both tasks were equally impaired at subanesthetic concentrations of isoflurane.

Emergence from anesthesia and return of cognitive function is faster using a combination of propofol and remifentanil as compared to desflurane and sevoflurane. Hence, the propofol-remifentanil combination has become increasingly popular among anesthesiologists.

Measuring saccadic eye movements is a reliable and sensitive method to assess residual effect general anesthesia Existing methods of measuring saccadic eye movement include electro-oculography (EOG) and use of high-speed video.

EOG has long-been available, and is probably the most widely used method for measuring horizontal eye movement in the clinic setting. EOG is a technique that can record a wide range of horizontal eye movements (±40°) but is less reliable for vertical eye movements. EOG uses the fact that a normal eyeball globe is an electrostatic dipole. The cornea is 0.4-1.0 mV positive relative to the opposite pole. Cutaneous electrodes are placed on both sides of the orbit. The potential difference recorded depends on the angle of the globe within the orbit.

Video detection of saccadic eye movements has been used. Normal video frame rates, however, of 30 Hz are slow relative to the high-speed saccade. Eye tracking devices, however, do exist for tracking this high-speed event using video speeds of 240 Hz or more. These devices are typically delicate and range in price from $10,000 to $40,000, and use high-speed cameras, delicate optics, CPU processors, image analysis software, and timed illumination sources to measure saccades.

### SUMMARY OF THE THE INVENTION

In general, in an aspect, the invention provides a saccadic-motion detector including an optical apparatus configured to focus light received from a subject's eye onto a focal plane, and an optical navigation chip comprising an optical sensing surface disposed substantially in the focal plane of the optical apparatus, the optical navigation chip configured to convert analog light reflected from the eye to digital indicia of movement of the eye.

Implementations of the invention may include one or more of the following features. The detector further includes a processor coupled to receive the digital indicia and configured to determine from the digital indicia a value indicative of a rate of movement of the eye. The rate includes at least one of speed and acceleration. The processor is configured to determine a condition associated with the subject based on the value of rate of movement of the eye. The processor is configured to compare the value of rate of movement of the eye with a table associating conditions and values of rate of movement of eyes to determine the subject's condition. The condition is at least one of normal, impaired, intoxicated, tired, dementia, delirium, psychosis, ADHD, depressed, and manic. The condition is impaired by at least one of benzodiazepines, narcotics, narcotic pharmaceutical mixtures, ethanol, barbiturates, and amphetamines.

Implementations of the invention may further include one or more of the following features. The optical navigation chip is configured to provide the digital indicia at a frequency above about 1200 Hz. The optical navigation chip is configured to provide the digital indicia at a frequency between about 1200 Hz and about 6000 Hz. The detector further includes a frame coupled to the optical apparatus and the optical navigation chip and configured to be grasped by a hand. The detector further includes a source of light configured to provide light to the eye to be reflected by the eye and received by the optical apparatus. The source of light is configured to provide near infrared light. The optical navigation chip comprises an array of charge coupled devices.

In general, in another aspect, the invention provides a system for detecting saccadic motion of a subject's eye, the system including a motion transducer, and an optical apparatus configured to focus light received from a subject's eye spanning a first aperture to a second aperture on an input of the motion transducer, the first aperture being larger than the second aperture, where the motion transducer is configured to capture a state of the focused light at different times and to provide at least one indication of least one of magnitude and direction differences in captured states of the light at the different times.

Implementations of the invention may include one or more of the following features. The system further includes a light source configured to provide light to the subject's eye, and a housing configured to hold the light source, the motion transducer, and the optical apparatus, where the housing includes a grip portion specifically configured to be grasped by a person's hand, and where the system is of a size and weight that make the system readily portable.

In general, in another aspect, the invention provides a system for detecting saccadic motion of a subject's eye, the system including a motion transducer configured to receive light at a first instance in time and a second instance in time indicative of a first position of the subject's eye and a second position of the subject's eye respectively and to provide at least one discrete indication of at least one of a magnitude and a direction difference between the first and second positions, and a processor coupled to the motion transducer and configured to process the at least one indication to determine a rate of movement of the subject's eye.

Implementations of the invention may include one or more of the following features. The motion transducer is configured to provide the indication at a frequency of at least about 1200 Hz. The motion transducer is configured to provide indicia of magnitude and direction differences in two dimensions. The at least one indication is one of a positive integer, a negative integer, and zero.

In general, in another aspect, the invention provides a method of processing saccadic eye movement information, the method including capturing a first state of light reflected from a subject's eye at a first time, capturing a second state of light reflected from a subject's eye at a second time, determining at least one of a magnitude difference and a direction difference between the first and second states, providing at least one indication of the at least one of a magnitude difference and a direction difference, and processing the at least one indication to determine a rate of movement of the subject's eye.

Implementations of the invention may include one or more of the following features. The method further includes providing an objective indication of a condition of the subject, the indicated condition being associated with a known rate that is similar to the determined rate. The method further includes comparing the determined rate with known rates and associated conditions. The first and second times are no more than about 1/1200^{th} of a second apart. The determining of the at least one of a magnitude difference and a direction difference comprises collapsing values of a two-dimensional array of values into at least one single dimension, and crosscorrelating multiple sets of collapsed data. The crosscorrelating comprises determining a direction and number of elements to shift a first set of collapsed values to best match with a second set of collapsed values.

Various aspects of the invention may provide one or more of the following capabilities. Objective measures of the residual motor and cognitive impairment after general anesthesia can be provided. Saccadic motion detection and analysis devices can be provided that are low cost, portable, durable, simple to use, battery powered, rugged, and/or robust. Saccadic motion detection and analysis can be employed to provide indicia of impairment due to drugs such as benzodiazepines, narcotics, narcotic pharmaceutical mixtures, ethanol, barbiturates, volatile gases, environmental toxins (e.g., sarin or mustard agents), and/or amphetamines. Saccadic motion detection and analysis can be employed to provide indicia of conditions such as fatigue, dementia, psychosis, attention deficit hyperactivity disorder (ADHD), depression, intoxication, sedation, and/or mania. Saccadic motion detection and analysis can be employed to provide indicia of degrees of impairment or influence of conditions. Patients that have undergone anesthesia may be monitored to determine their level of anesthesia over time. Patients can be diagnosed with conditions such as neurological defects, or indicia of characteristics associated with neurological conditions. Money can be saved, e.g., by avoiding costs of urine, blood, and/or hair samples and testing, and because per-test cost can be small. Medical diagnosis can be provided in a safe, non-invasive manner (e.g., without phlebotomies or urine samples). Saccadic motion analysis can be provided in an automated manner. Training of personnel to analyze saccadic motion can be reduced and/or eliminated. Saccadic motion can be measured, analyzed, and reported in a short amount of time, e.g., one minute. Saccadic motion analysis can be provided in a reliable manner, e.g., avoiding risk of sample switching or loss. A broad spectrum of conditions related to saccadic motion can be diagnosed. Conditions can be determined in a manner that is difficult to circumvent or defeat.

These and other capabilities of the invention, along with the invention itself, will be more fully understood after a review of the following figures, detailed description, and claims.

### BRIEF DESCRIPTION OF THE FIGURES

FIG. 1 is a schematic diagram of a method and system of measuring saccade motion.

FIGS. 2-3 are simplified exemplary depictions of captured light by a four by four array of light-sensitive elements.

FIG. 4 is a photograph of an exemplary saccade motion detector.

FIG. 5 is a block flow diagram of a process of measuring saccade motion and determining a condition associated with the measured motion.

### DETAILED DESCRIPTION OF PREFERRED EMBODIMENTS

Embodiments of the invention provide techniques for measuring saccade motion and determining a condition corresponding to the measured saccade motion. Light from an eye is focused onto an aperture of an optical navigation chip. This chip employs a charge-coupled device (CCD) array to measure the incident light and digitize the incident analog light. The collection of amounts of light measured at the various elements of the array form a state of the incident light, with the state being related to a position of the eye. States of the incident light are measured by the CCD array at frequencies of 1200 Hz or more (e.g., 6000 Hz). The chip processes multiple states of the CCD array to determine changes between states. The chip determines magnitudes and directions of change of the states in two axes, e.g., x and y. The chip quantifies the magnitude into an integer and the direction into a polarity. The changes are reported by the chip to a processor that analyzes the reported changes to determine rates (e.g., speeds and/or accelerations) of motion of the eye. These rates are compared by the processor to known rates and their associated conditions (e.g., that the subject is anesthetized, impaired, fatigued, intoxicated, etc.). The processor provides an indication of the determined condition associated with the rate of motion of the eye. While this description reflects exemplary currently-preferred embodiments, other embodiments are within the scope of the invention.

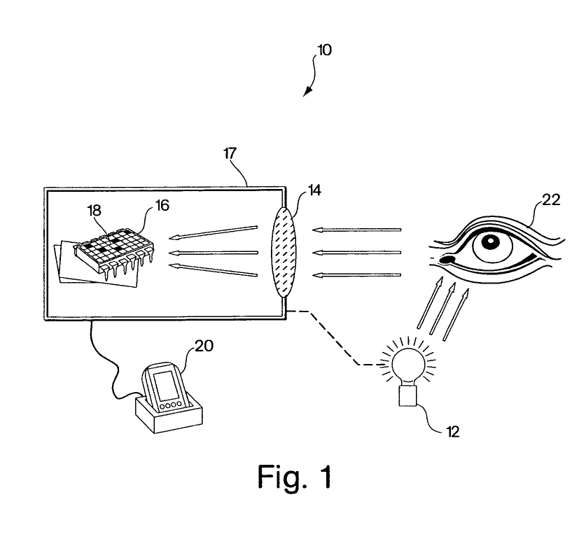

Referring to FIG. 1, a saccadic eye movement detection and analysis system 10 includes a light source 12, an optical focusing apparatus, here a lens, 14, a light sensor 16 that includes a photosensitive array 18, and a processor 20. The lens 14 and the light sensor 16 are contained by a housing 17, shown schematically in FIG. 1 as a box. The light source 12 is also preferably contained by the housing 17 as indicated by a dotted line in FIG. 1, but is shown separately in FIG. 1 for illustrative purposes. The system 10 is configured to detect and analyze saccadic motion of a subject's eye 22. The system 10 utilizes optical tracking technology combined with simple optics to capture and analyze images from the eye 22. The light sensor 16 is connected to the processor 20 for electrical communication, e.g., with a universal serial bus (USB) line or other communications connection. The processor 20 can be any of a variety of devices and have a variety of forms. As shown in FIG. 1, the processor is a personal digital assistant (PDA). The system 10 is preferably configured to be of a size and weight that make the system 10 readily portable, e.g., hand-carried by a single person. For example, the system 10 may weigh less than about 1 pound, and have dimensions of about 1×3×9 inches so that it is readily handled with one hand, and is near the size of an ophthalmoscope.

The light source 12 is configured to illuminate a surface of the patient's eye 22. The light source 12 can be any of a variety of light sources that preferably provide light in wavelengths within a range from visible light to infrared. As shown, here the light source 12 is a light bulb, although other devices, e.g., a light emitting diode (LED) would be acceptable. The light source 12 is preferably configured as a handheld device or other easily portable, movable device.

The lens 14 is configured to capture and focus light from the eye 22. Reflected and/or scattered light from, e.g., the conjuctiva, sclera and/or cornea of the eye 22 is captured by the optical apparatus 14 located near the patient's eye 22 (but preferably not touching the eye 22). Light from the surface of the eye 22 is brought to focus by the apparatus 14 on the light sensor 16, and preferably on a light-sensitive surface and/or region of the sensor 16. The lens 14 is preferably configured to focus light from the source 12 that is reflected by the entire width and height of the eye 22 to the entire length and width of the photosensitive array 18 such that light reflected by the extremes of the eye is detected by the extremes of the array 18, or a representative patch of the corneal surface is reflected to the entire length and width of the photosensitive array 18.

The light sensor 16 is preferably an optical navigation semiconductor chip 16 containing the photosensitive array 18. For example, the sensor 16 can be the photosensitive chip Model ADNS-2620 made by Agilent Technologies of Palo Alto, Calif. The Agilent ADNS-2620 is a small form-factor optical mouse sensor that is produced in high volume and underlies the non-mechanical tracking engine for many computer mice. This optical navigation technology measures changes in position by optically acquiring sequential surface images (frames) and mathematically determining the direction and magnitude of movement. There are no moving parts, so precision optical alignment is not required, thereby facilitating high volume assembly. The array 18 comprises a two-dimensional set, e.g., 16 by 16, of CCD elements 24 to provide 256 pixels of captured light. The elements 24 are configured to provide indicia of intensity of light received by each element. The elements 24 are clocked to provide their indicia of collected light (e.g., charge produced by the received light), thereby emptying their stored charge and resetting their wells for more light capture. The elements 24 can provide rapid image capture, e.g., rates from about 1200 Hz to about 6000 Hz or more. These frequencies are exemplary only, and other capture frequencies may be used, including frequencies less than 1200 Hz, e.g., 600 Hz, and more than 6000 Hz.

The system 10 uses an optimization schema based on mathematical cross-correlations of sequential frames to determine movement. The chip 16 analyzes successive states of the array, e.g., separated in time by from about 1/6000^{th} of a second to about 1/1200^{th} of a second, to determine whether a significant difference in the values detected by elements of the array 18 exists. The system uses an optimization schema described in the sequel. Fractions of pixels are ignored. If the image was perfectly random from frame to frame, the system generate random numbers from the set {−7, −6, −5, −4, −3, −2, −1, 0, 1, 2, 3, 4, 5, 6, 7} independently in the X and Y channels.

If not, then the chip 16 can output nothing, or an indication of no significant change to the processor 20. If a significant change has occurred, then the chip 16 determines two values indicative of the change. These two values represent the magnitude and direction of the changes in the two dimensions of the array 18, e.g., x and y. For example, if the array 18 is a 16 by 16 array, then the chip preferably can output two values each ranging between −7 and +7, the number indicating the magnitude of the change and the polarity indicating the direction of the change. This processing preferably occurs on the chip 16 so that a host computer 20 is spared the notorious and computationally intense problem of image recognition. The chip 16 is preferably silent when there is no change and when movement occurs data is reported in standard USB format. The technology is precise and high-speed, making it well-suited for monitoring eye motion. In the case of computer mice, the data are reported as a rapid series of integer xy data that is processed to give the sensation of fluid, continuous motion of the mouse pointer.

To represent eye motion, the data are processed by an optimization schema using cross correlation. For example, the array 18 may be a 16 by 16 array of elements. To determine the x data, the columns (y-direction) are collapsed by combining (e.g., averaging) the values of all the elements in each column to produce a single row (x-direction) set of 16 values. This is done for multiple frames from the array 18 at different times. Two single-row sets are compared (cross correlated) to determine which direction and magnitude shift of set 1 (preferably the earlier-in-time set) will best map set 1 to set 2. For a 16 by 16 array, and thus a 16-element row, set 0 can be shifted up to 7 elements in either direction, thus yielding x data in the range of −7 to +7. The same procedure is applied to determine the y data (collapsing rows into a single column and cross correlating single columns of data).

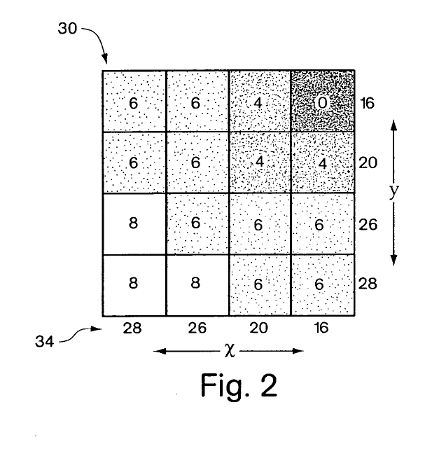

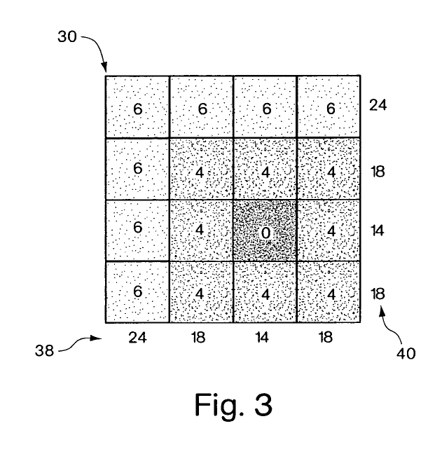

Referring also to FIGS. 2-3, the light sensor chip 16 is configured to analyze multiple (e.g., successive) images captured by the array 18 to perform the cross correlation. For exemplary purposes only, a four by four array 30 is shown at two different times, t₁ and t₂, corresponding to FIGS. 2 and 3, respectively. As shown, the detected intensity of light has numerical intensities corresponding to lightness/darkness. Here, for simplicity, the intensities are shown as being one of four values 8, 6, 4, 0 for each pixel, with 8 being for the highest intensity, lightest detectable level of light and 0 being for the lowest intensity, darkest level of light. The darkest area typically corresponds to the location of the pupil in the eye 22 as the pupil absorbs more light from the source 12 than other areas of the eye 22. As shown in FIG. 2, at time t₁ the pupil likely corresponds to the top right of the detected region of light. The chip 16 is configured to collapse the detected intensities in the x and y directions. Thus, collapsing the columns down to the x-axis by adding the intensities in the columns yields x-values 34 of 28, 26, 20, and 16 (with averages of 7.5, 6.5, 5, and 4) and collapsing the rows to the y-axis by adding the intensities in the rows yields y-values 36 of 16, 20, 26, and 28 (averages of 4, 5, 6.5, and 7.5). Performing the same collapsing for the array 30 shown in FIG. 3 at time t₂ yields x-values 38 collapsed to the x-axis of 24, 18, 14, and 18 (6, 4.5, 3.5, and 4.5 averages) and y-values 40 collapsed to the y-axis of 24, 18, 14, and 18 (6, 4.5, 3.5, and 4.5 averages).

The light sensor chip 16 is configured to compare the intensity distributions in different images to determine magnitude and direction of eye (e.g., pupil) movement. In FIGS. 2-3, the darkest pixel moves from the upper right comer of the array 30 in FIG. 2 down two pixels in the negative y-direction and left one pixel in the negative x-direction. The chip 16 can determine this motion by comparing the x and y distributions of intensities in the two images. The x-distribution 34 of 28, 26, 20, 16 from FIG. 2 can best match the x-distribution 38 of 24, 18, 14, 18 from FIG. 3 by moving the FIG. 2 distribution to the left one pixel to yield 26, 20, 16, ?. Similarly, shifting the y-distribution 36 of 16, 20, 26, 28 down two slots to yield?,?, 16, 20 best matches with the y-distribution 40 of 24, 18, 14, 18 of FIG. 3. The shifted distributions best match where they have less deviation from the corresponding intensities of the new image than any other shifted distribution. A weighting schema can be included, for example, such that pixels near the center would be given more weight or importance that peripheral pixels. The light sensor 16 would thus output (−1, −2) as the movement between the images shown in FIGS. 2 and 3. Knowing the x and y shifts, in this example, −1 in the x-direction and −2 in the y-direction, the chip 16 can send the x and y movement data to the processor 20.

The information reported by the light sensor 16 is a rapid series of integer x-y data that is processed to represent eye motion. The processor 20 is configured with appropriate processing capability (e.g., a CPU) and memory for storing software instructions to be executed by the CPU to determine desired quantities. In particular, the processor 20 is configured to use the received x-y data and the processor's internal clock to determine velocities, accelerations, directions, latencies, and the like of the eye 22. For example, for the two images shown in FIGS. 2-3, if one pixel is 1 mm by 1 mm, and the frames shown in FIGS. 2 and 3 were taken 1/6000^{th} of a second apart, then the speed of the eye 22 can be determined according to:

$$\begin{matrix}Speed = \frac{distance}{time} = ( {( {( 1 )}^{2} + {( - 2 )}^{2} )}^{1 / 2} ) / ( 1 / 6000 ) \\ = 13 , 416 mm \text{/} s \\ = 13.4 m \text{/} s\end{matrix}$$

The processor 20 also includes software code for processing, scoring, and presenting the data, so as to address issues such as internal validity or statistical significance. Motion information sensed and/or determined by the sensor 16 and/or the processor 20 can be selectively presented. For example, information can be filtered to eliminate data indicative of non-saccadic motion (e.g., turning of the subject's head). Further, saccadic motion information may be presented as, and/or in conjunction with, indications of conditions associated with the saccadic motion. For example, the processor 20 can compare the sensed/determined saccadic motion information with known relationships of saccadic motion values and corresponding conditions such as intoxication, fatigue, anthesthetized, etc. If the determined saccadic motion matches, or at least is acceptably similar to (e.g., within a tolerance of), a saccadic motion value for which a corresponding condition is known, then the processor 20 can report the condition. For example, if the system 10 is adapted for use by police officers for field sobriety tests, the processor 20 could provide an objective indication of whether the subject is intoxicated, and possibly to what extent, or that the subject is suffering from some other condition such as dementia or fatigue, or that the subject's saccadic motion indicates that the subject is not suffering from any abnormal condition.

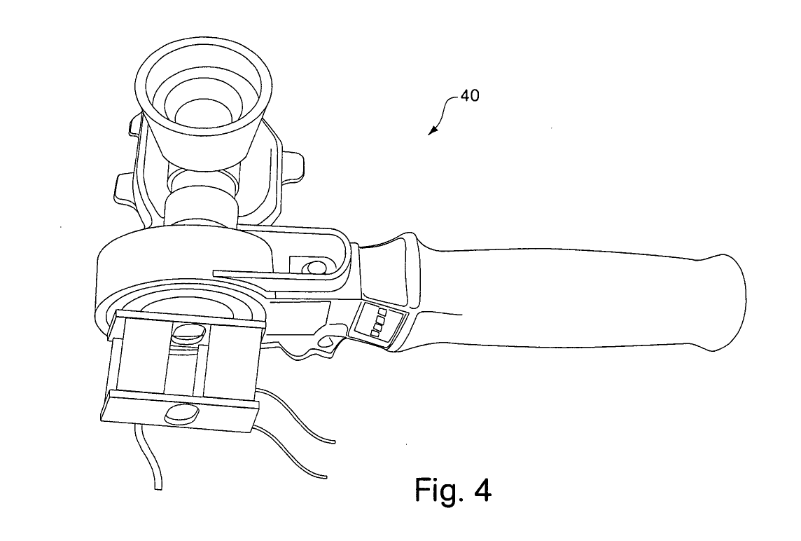

As shown in FIG. 4, in an exemplary embodiment, a handheld device 40 includes a light source, a focusing apparatus, and a light sensor contained by a housing. The handheld device 40 is small and portable. The housing includes a handle that is configured to be grasped manually. The device 40 is relatively lightweight and can be battery operated to facilitate its portability. It can be removably connected to the computer 20. The device 40 can be used when separated from the computer 20, store its measured data and transfer the stored data to the computer 20 when linked to the computer 20 for communication (either through a physical connection or remotely). Alternatively, the device 40 could be configured to wirelessly communicate the processor 20, or even include the processor 20.

The system 10 is also adaptable. The software and/or hardware and/or firmware of the light sensor 16, the computer 20, and/or of the device 40 can be upgraded to adapt to new technologies, new associations of saccadic motion to conditions, etc.

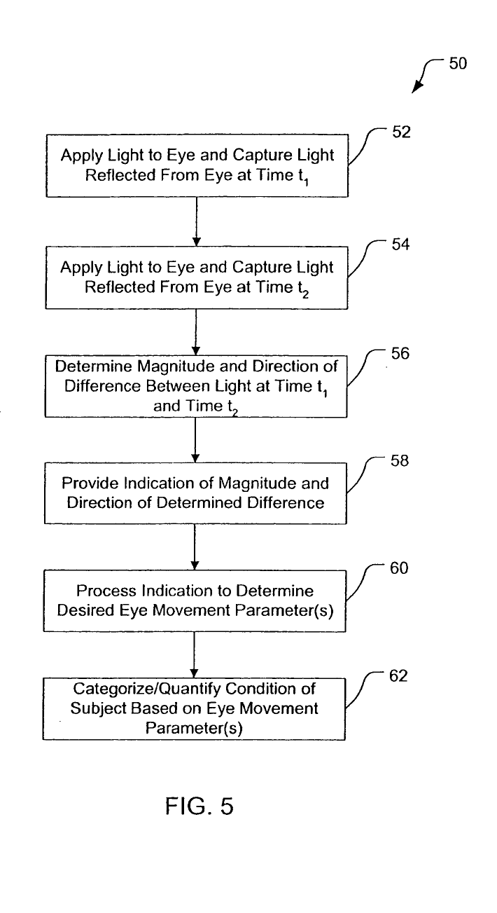

In operation, referring to FIG. 5, with further reference to FIGS. 1-3, a process 50 for sensing, determining, and categorizing saccadic eye movement using the system 10 includes the stages shown. The process 10, however, is exemplary only and not limiting. The process 50 may be altered, e.g., by having stages added, removed, or rearranged.

At stage 52, at a first time t₁ light is applied to the eye 22 and light reflected from the eye 22 is captured. The light source 12 is actuated, e.g., by the processor 20 (with the processor 20 coupled to the source 12 and configured to actuate the source 12), or manually by an operator. The light is directed at the subject's eye 22, e.g., by an operator manipulating the housing 17 to aim the light source 12 at the eye 22. Light reflects from the eye 22 and is captured by the sensor 16 through the array of photosensitive elements 18 as a first image.

At stage 54, at a second time t₂ light is applied to the eye 22 and light reflected from the eye 22 is captured. As at stage 52, the light source 12 is actuated, e.g., by the processor 20 (with the processor 20 coupled to the source 12 and configured to actuate the source 12), or manually by an operator. The light is directed at the subject's eye 22, e.g., by an operator manipulating the housing 17 to aim the light source 12 at the eye 22. Light reflects from the eye 22 and is captured by the sensor 16 through the array of photosensitive elements 18 as a second image.

At stage 56, magnitude and direction differences between the image captured at the first time t₁ and the second time t₂ are determined. The light sensor 16 analyzes the intensities of the captured images. As discussed above, the sensor 16 can collapse the intensities in x- and y-dimensions for both images and compare the collapsed intensities of the two images. From this comparison, the sensor 16 determines the amounts and directions of shifts to the x- and y-values of the first image that will cause the collapsed intensities of the first image to best match the collapsed intensities of the second image.

At stage 58, the light sensor 16 provides indications of the magnitude and directional shifts to best match the first image to the second image. These indications may be provided even if no shifts are in order, but preferably are provided only if a magnitude shift at least as great as a threshold amount is in order, including if the shift is a total distance shifted even if not above the threshold in the x- or y-direction alone. Thus, the times t₁ and t₂ may be consecutive times of the sensor 16, e.g., consecutive clockings of the sensor 16, if all clockings are reported regardless of shift magnitudes, or it there is a significant shift in order between consecutive clockings. The times t₁ and t₂ may, however, be nonconsecutive times separated by one or more clock cycles. For example, the second time may correspond to a clock cycle where the light sensor 16 determines that a change in the captured image above a threshold amount has occurred relative to the image captured at the first time, but where this change does not occur between consecutive clock cycles of the sensor 16.

At stage 60, the processor 20 uses the indications provided by the light sensor 16 regarding image shifts to determine one or more eye movement parameters. The processor 20 uses the magnitudes and directions of shifts indicated by the sensor 16 to calculate or otherwise determine (e.g., using a lookup table or a polynomial curve fit that maps these magnitudes and directions to eye motion. distance of movement of the eye 22, e.g., of the pupil or other anatomical feature of the eye. Further, the processor 20 determines the time used by the eye 22 to move the determined distance, e.g., by analyzing times of arrival of current and previous indications of movement, or by analyzing an indication of time of movement provided by the light sensor 16 or indications of the two times t₁ and t₂ provided by the sensor 16, etc. From the indications of time and distance of one or more movements by the eye 22, the processor can determine various parameters such as velocity, acceleration, latency, etc. associated with the eye movement(s).

At stage 62, the processor 20 categorizes and possibly quantifies a condition associated with the eye movement. The processor 20 relates the determine parameter(s) to known relationships between parameters and conditions to attempt to categorize the condition associated with the movement, e.g., anesthetized, intoxicated, delusional, etc. The processor 20 may also attempt to quantify conditions, e.g., heavily anesthetized, lightly anesthetized, anesthetized sufficiently/insufficiently for a particular procedure, sufficiently de-anesthetized, legally intoxicated, etc. The processor 20 may actuate various indicators, e.g., lights or other visual indicators (e.g., LCDs, readouts, etc.), audible indicators such as tones, etc. to indicate the subject's condition and possibly severity of that condition to an operator of the system 10. For example, the processor 20 may indicate that the subject's condition is normal, impaired, intoxicated, tired, dementia, delirium, psychosis, ADHD, depressed, and manic. The impaired condition may be due to use by the subject of one or more substances such as benzodiazepines, narcotics, narcotic pharmaceutical mixtures, ethanol, barbiturates, and amphetamines. The system 10 may determine whether a stimulant, such as amphetamine, or a depressant such as alcohol is present; for example, the system 10 may be adapted to detect opiate use, as overly-constricted pupils is quite specific for opiate use/abuse.

The process 50 allows the invention to be used for a wide variety of applications. The invention can be used for objective measurement of recovery from anesthesia after a medical/surgical procedure. The invention can be applied to a variety of medical and non-medical disciplines, such as anesthesia, emergency medicine, neurology, psychiatry, critical care, ophthalmology, geriatrics, forensic medicine, alcohol intoxication, drug intoxication, drug compliance, impairment due to fatigue, etc.

These areas may find numerous specific uses for the invention. For anesthesiologists and/or intensivists may find use for the invention, e.g., in the post anesthesia care unit (PACU), for critical care (e.g., ICU), in pain management clinics, in operating rooms, in hospitals generally, for diagnosing dementia vs. delirium, and for pre-op screening including substance abuse. Neurologists may use the invention for diagnosing and/or monitoring conditions such as multiple sclerosis, myasthenia gravis, ALS, Alzheimer's, stroke (CVA), time course of brain disease, and substance abuse. Opthamalogists and otolaryngologists (ENTs) may use the invention for monitoring and/or diagnosing motor vs. visual defects, strabismus and nystagmus, vertigo and vestibular function, and trauma. Psychiatrists may use the invention for diagnosing and/or monitoring dementia vs. delirium, mood disorders vs. psychosis, and substance abuse. Emergency room personnel may use the invention for diagnosing and/or monitoring delerium vs. dementia, stroke (CVA), trauma, environmental toxin (including terrorism toxin) influence, and substance abuse. Any of these or other people may find further uses for the invention, and these uses noted are exemplary only and not limiting.

The invention can be applied to needs in the hospital, clinic and day surgery center, forensic testing and law enforcement and public safety agencies. The invention may also be used for lay operators, and/or for commercial (e.g., job site) and/or home use (e.g., by parents or guardians). The invention may be used for numerous forensic applications. Manufacturers that use or make explosives or flammable materials, or whose personnel use heavy equipment, may use the invention, e.g., to detect intoxication of employees. Mission critical enterprises such as financial and accounting institutions, or employers of computer programmers or operators may likewise use the invention, e.g., for detecting intoxicated employees. For similar reasons, and others, transportation providers such as airline carriers, bus operators, car rental companies, taxi/limousine/livery companies, ship operators, etc. may also use the invention. Other applications are also within the scope of the invention, both for listed and unlisted users.

Other embodiments are within the scope and spirit of the appended claims. For example, due to the nature of software, functions described above can be implemented using software, hardware, firmware, hardwiring, or combinations of any of these. Also, the techniques discussed above regarding calculating motion (see FIGS. 2-3 and discussion) are exemplary and not limiting; Other techniques for calculating motion may be used. Features implementing functions may also be physically located at various positions, including being distributed such that portions of functions are implemented at different physical locations. For example, functions described above being performed in the chip 16 may be partially or entirely performed in the processor 20, and functions described above being performed in the processor 20 may be partially or entirely performed in the chip 16. Also, devices other than optical navigation semiconductor chips may be used to detect and transduce light from the eye. Also, while the computer 20 is shown in FIG. 1 as a personal digital assistant (PDA), other forms of computing devices are acceptable, such as personal desktop or laptop computers, etc. The computer 20 may be hardwired to the apparatus 13, or may be wirelessly connected to the device 13, e.g., using Bluetooth, or IEEE 802.11b protocols. Further, embodiments of the invention may not use a focusing apparatus light the apparatus 14 shown in FIG. 1. Light reflected from the eye 22 could be captured by the light sensor 16 without having been focused on the sensor 16. Further still, the light sensor 16 may output indicia of the current state of sensed light (e.g., since last clocking) whether or not a significant change has occurred.

1. A saccadic-motion detector comprising:
an optical apparatus configured to focus light received from a subject's eye onto a focal plane; and
an optical navigation chip comprising an optical sensing surface disposed substantially in the focal plane of the optical apparatus, the optical navigation chip configured to convert analog light reflected from the eye to digital indicia of movement of the eye.

2. The detector of claim 1 further comprising a processor coupled to receive the digital indicia and configured to determine from the digital indicia a value indicative of a rate of movement of the eye.

3. The detector of claim 2 wherein the rate includes at least one of speed and acceleration.

4. The detector of claim 2 wherein the processor is configured to determine a condition associated with the subject based on the value of rate of movement of the eye.

5. The detector of claim 4 wherein the processor is configured to compare the value of rate of movement of the eye with a table associating conditions and values of rate of movement of eyes to determine the subject's condition.

6. The detector of claim 4 wherein the condition is at least one of normal, impaired, intoxicated, tired, dementia, delirium, psychosis, ADHD, depressed, and manic.

7. The detector of claim 6 wherein the condition is impaired by at least one of benzodiazepines, narcotics, narcotic pharmaceutical mixtures, ethanol, barbiturates, and amphetamines.

8. The detector of claim 1 wherein the optical navigation chip is configured to provide the digital indicia at a frequency above about 1200 Hz.

9. The detector of claim 8 wherein the optical navigation chip is configured to provide the digital indicia at a frequency between about 1200 Hz and about 6000 Hz.

10. The detector of claim 1 further comprising a frame coupled to the optical apparatus and the optical navigation chip and configured to be grasped by a hand.

11. The detector of claim 1 further comprising a source of light configured to provide light to the eye to be reflected by the eye and received by the optical apparatus.

12. The detector of claim 11 wherein the source of light is configured to provide near infrared light.

13. The detector of claim 1 wherein the optical navigation chip comprises an array of charge coupled devices.

14. A system for detecting saccadic motion of a subject's eye, the system comprising:
a motion transducer; and
an optical apparatus configured to focus light received from a subject's eye spanning a first aperture to a second aperture on an input of the motion transducer, the first aperture being larger than the second aperture;
wherein the motion transducer is configured to capture a state of the focused light at different times and to provide at least one indication of least one of magnitude and direction differences in captured states of the light at the different times.

15. The system of claim 14 further comprising:
a light source configured to provide light to the subject's eye; and
a housing configured to hold the light source, the motion transducer, and the optical apparatus;
wherein the housing includes a grip portion specifically configured to be grasped by a person's hand; and
wherein the system is of a size and weight that make the system readily portable.

16. A system for detecting saccadic motion of a subject's eye, the system comprising:
a motion transducer configured to receive light at a first instance in time and a second instance in time indicative of a first position of the subject's eye and a second position of the subject's eye respectively and to provide at least one discrete indication of at least one of a magnitude and a direction difference between the first and second positions;
a processor coupled to the motion transducer and configured to process the at least one indication to determine a rate of movement of the subject's eye.

17. The system of claim 16 wherein the motion transducer is configured to provide the indication at a frequency of at least about 1200 Hz.

18. The system of claim 16 wherein the motion transducer is configured to provide indicia of magnitude and direction differences in two dimensions.

19. The system of claim 16 wherein the at least one indication is one of a positive integer, a negative integer, and zero.

20. A method of processing saccadic eye movement information, the method comprising:
capturing a first state of light reflected from a subject's eye at a first time;
capturing a second state of light reflected from a subject's eye at a second time;
determining at least one of a magnitude difference and a direction difference between the first and second states;
providing at least one indication of the at least one of a magnitude difference and a direction difference; and
processing the at least one indication to determine a rate of movement of the subject's eye.

21. The method of claim 20 further comprising providing an objective indication of a condition of the subject, the indicated condition being associated with a known rate that is similar to the determined rate.

22. The method of claim 21 further comprising comparing the determined rate with known rates and associated conditions.

23. The method of claim 20 wherein the first and second times are no more than about 1/1200^{th} of a second apart.

24. The method of claim 20 wherein the determining of the at least one of a magnitude difference and a direction difference comprises collapsing values of a two-dimensional array of values into at least one single dimension, and crosscorrelating multiple sets of collapsed data.

25. The method of claim 24 wherein the crosscorrelating comprises determining a direction and number of elements to shift a first set of collapsed values to best match with a second set of collapsed values.
